(19) United States
(12) Patent Application Publication
(10) Pub. No.: US 2006/0246210 A1
(43) Pub. Date: Nov. 2, 2006

(54) Method for making a covered drug-eluting stent

(75) Inventors: Kobi Iki, San Carlos, CA (US); Marshall Tsuruda, San Jose, CA (US); Teodoro C. Tecson, Santa Clara, CA (US); Angelica Alvarado, Morgan Hill, CA (US); Jasbir K. Badesha, Milpitas, CA (US)

Correspondence Address: HAYNES BEFFEL & WOLFELD LLP, P O BOX 366, HALF MOON BAY, CA, 94019, US

(73) Assignee: VASCULAR ARCHITECTS INC., a Delaware Corporation, San Jose, CA (US)

(21) Appl. No.: 11/119,367

(22) Filed: Apr. 29, 2005

(51) Int. Cl. A61L 33/00

(52) U.S. Cl. 427/2.25

(57) ABSTRACT

A stent, having openings, is mounted onto a mandrel. An agent-containing film is applied onto the stent and the two are pressed against one another so that at least a portion of the film is pressed at least partially into the openings. The film is adhered to the stent. Any excess film is removed to create a stent/film combination which is removed from the mandrel and enclosed within a sleeve of porous material to create a covered agent-eluting stent.

### CROSS-REFERENCE TO OTHER APPLICATIONS

This is related to the following: U.S. patent application Ser. No. 09/740,597 filed Dec. 19, 2000; U.S. patent application Ser. No. 09/910,703 filed Jul. 20, 2001; U.S. Pat. No. 6,248,122 B1 issued Jun. 19, 2001; U.S. Pat. No. 6,238,430 issued May 29, 2001; U.S. Pat. No. 6,645,237 issued Nov. 11, 2003; U.S. Pat. No. 6,572,648 issued Jun. 3, 2003; and U.S. patent application Ser. No. 10/941,064 filed Sep. 14, 2004.

### FEDERALLY SPONSORED RESEARCH OR DEVELOPMENT

None.

### BACKGROUND OF THE INVENTION

The present invention provides for the delivery of a therapeutic agent by a covered stent to a target site within a hollow body structure of the patient, particularly within the vascular system for the treatment of cardiovascular and peripheral vascular disease, such as vascular stenoses and restenoses, dissections and other tissue separation conditions, aneurysms, and the like.

Research has been done to determine the causes and possible treatments of coronary restenosis following balloon angioplasty. Restenosis following balloon angioplasty is believed to result from several causes, including elastic recoil of the vessel, thrombus formation and cell wall growth. The article, Chan, A W, Chew, D P, and Lincoff A M, Update on Pharmacology for Restenosis, Current Interventional Cardiology Reports 2001, 3:149-155, concludes that restenosis remains a major problem for percutaneous coronary intervention and that while drug-eluting stents may be found to be effective, larger clinical trials are needed.

The apparatus of the present invention, however, are also useful for placement in other hollow body structures, such as the ureter, urethra, bronchus, biliary tract, gastrointestinal tract and the like, for the treatment of other conditions which may benefit from the introduction of a therapeutic agent along with a reinforcing or protective structure within the body lumen. The prostheses will typically be placed endoluminally. As used herein, “endoluminally” will mean placement by percutaneous or cutdown procedures, wherein the prosthesis is transluminally advanced through the body lumen from a remote location to a target site in the lumen. In vascular procedures, the prostheses will typically be introduced “endovascularly” using a catheter over a guidewire under fluoroscopic, or other imaging system, guidance. The catheters and guidewires may be introduced through conventional access sites to the vascular system, such as through the femoral artery, or brachial and subclavian arteries, for access to the target site.

An endoluminal prosthesis typically comprises at least one radially expansible, usually cylindrical, body segment. By “radially expansible,” it is meant that the body segment can be converted from a small diameter configuration (used for endoluminal placement) to a radially expanded, usually cylindrical, configuration which is achieved when the prosthesis is implanted at the desired target site. The prosthesis may be non-resilient, e.g., malleable, thus requiring the application of an internal force to expand it at the target site. Typically, the expansive force can be provided by a balloon catheter, such as an angioplasty balloon for vascular procedures. Alternatively, the prosthesis can be self-expanding. Such self-expanding structures may be provided by a temperature-sensitive superelastic material, such as Nitinol, which naturally assumes a radially expanded condition once an appropriate temperature has been reached. The appropriate temperature can be, for example, a temperature slightly below normal body temperature; if the appropriate temperature is above normal body temperature, some method of heating the structure must be used. Another type of self-expanding structure uses resilient material, such as a stainless steel or superelastic alloy, and forming the body segment so that it possesses its desired, radially-expanded diameter when it is unconstrained, e.g., released from radially constraining forces of a sheath. To remain anchored in the body lumen, the prosthesis will remain partially constrained by the lumen. The self-expanding prosthesis can be delivered in its radially constrained configuration, e.g. by placing the prosthesis within a delivery sheath or tube and retracting the sheath at the target site. Such general aspects of construction and delivery modalities are well-known in the art.

The dimensions of a typical endoluminal prosthesis will depend on its intended use. Typically, the prosthesis will have a length in the range from 0.5 cm to 15 cm, usually being from about 0.8 cm to 10 cm, for vascular applications. The small (radially collapsed) diameter of cylindrical prostheses will usually be in the range from about 1 mm to 10 mm, more usually being in the range from 1.5 mm to 6 mm for vascular applications. The expanded diameter will usually be in the range from about 2 mm to 50 mm, preferably being in the range from about 3 mm to 15 mm for vascular applications and from about 25 mm to 45 mm for aortic applications.

One type of endoluminal prosthesis includes both a stent component and a covering component. These endoluminal prostheses are often called stent grafts or covered stents. A covered stent is typically introduced using a catheter with both the stent and covering in contracted, reduced-diameter states. Once at the target site, the stent and covering are expanded. After expansion, the catheter is withdrawn from the vessel leaving the covered stent at the target site. Coverings may be made of, for example, PTFE, ePTFE or Dacron® polyester.

Grafts are used within the body for various reasons, such as to repair damaged or diseased portions of blood vessels such as may be caused by injury, disease, or an aneurysm. It has been found effective to introduce pores into the walls of the graft to provide ingrowth of tissue onto the walls of the graft. With larger diameter grafts, woven graft material is often used. You get grades including a three-month In small and large diameter vessels, porous fluoropolymers, such as ePTFE, have been found useful.

Coil-type stents can be wound about the catheter shaft in torqued compression for deployment. The coil-type stent can be maintained in this torqued compression condition by securing the ends of the coil-type stent in position on a catheter shaft. The ends are released by, for example, pulling on wires once at the target site. See, for example, U.S. Pat. Nos. 5,372,600 and 5,476,505. Alternatively, the endoluminal prosthesis can be maintained in its reduced-diameter condition by a sleeve; the sleeve can be selectively retracted to release the prosthesis. A third approach is the most common. A balloon is used to expand the prosthesis at the target site. The stent is typically extended past its elastic limit so that it remains in its expanded state after the balloon is deflated and removed. One balloon expandable stent is the Palmaz-Schatz stent available from the Cordis Division of Johnson & Johnson. Stents are also available from Medtronic AVE of Santa Rosa, Calif. and Guidant Corporation of Indianapolis, Ind.

### BRIEF SUMMARY OF THE INVENTION

The present invention is directed to a method for making a covered agent-eluting stent. A stent having a stent body with openings formed therein is obtained. The stent is mounted onto a mandrel. An agent-containing film is applied onto the stent mounted on the mandrel to create a first subassembly. The stent and the film are pressed against one another to create a second subassembly with at least a portion of the film pressed at least partially into the openings of the stent body. The film is adhered to the stent. Any excess film is removed from the second subassembly to create a stent/film combination having inner and outer surfaces. The stent/film combination is removed from the mandrel and the stent/film combination is enclosed within a sleeve of porous material to create a covered agent-eluting stent.

Some method according to the invention may include one, some or all of the following. A coiled stent having axially spaced-apart turns to define a generally helical gap between the turns may be used. A strip of the agent-containing film may be wound onto the stent. A diffusion restrictor may be applied on the outer surface of the stent/film combination, the diffusion restrictor permitting passage of the agent through the diffusion restrictor at a first, therapeutic rate. A diffusion barrier may be applied on the inner surface of the stent/film combination, the diffusion barrier preventing passage of the agent through the diffusion barrier at at most a second rate, the second rate being less than the first rate. A bolus-creating agent-containing material may be applied on the diffusion restrictor.

The agent may be part of a therapeutic agent/silicone carrier matrix secured to, that is adhered to or otherwise in intimate contact with, the stent body. The therapeutic agent may comprise a hydrophilic anti-restenosis drug, preferably at least one of Sodium Nitroprusside, L-Arginine or Poly L-Arginine. Thus, the invention provides for the controlled, stent-based release of a hydrophilic compound using a covered stent in a vascular/aqueous environment. The diffusion restrictor and the diffuser barrier may both comprise Parylene. The diffusion barrier may be a substantially non-porous vapor-deposited layer of Parylene and the diffusion restrictor may be a micro-porous vapor-deposited layer of Parylene.

Various features and advantages of the invention will appear from the following description in which the preferred embodiments have been set forth in detail in conjunction with the accompanying drawings.

### BRIEF DESCRIPTION OF THE DRAWINGS

FIG. 1 illustrates a conventional ladder type stent blank;

FIG. 2 illustrates the stent blank of FIG. 1 formed into a generally helical coil;

FIG. 3 shows a covered stent including a coiled stent as in FIG. 2 covered by a sleeve of material;

FIG. 4 is a cross sectional view of a stent blank taken along line 4-4 of FIG. 1;

FIG. 5 shows the stent of FIG. 4 after a silicone/therapeutic matrix material has been applied thereto;

FIG. 6 illustrates the application of a diffusion barrier material to an inner stent body surface of the structure of FIG. 5;

FIG. 7 illustrates the application of a diffusion restrictor material to an outer stent body surface of the stent of FIG. 6 to create a stent structure;

FIG. 8 shows the stent structure of FIG. 7 after being covered with a sleeve of porous material;

FIG. 9 is a simplified cross sectional view of a covered stent, similar to that of FIG. 8 with the various layers separated for purposes of illustration, positioned within a vessel and against the vessel wall;

FIG. 10 is an overall view of an alternative stent body made of expanded metal;

FIG. 11 shows an alternative to the covered ladder stent of FIG. 3;

FIGS. 12-18 illustrate an alternative method for making a covered agent-eluting stent with FIG. 12 being a flowchart showing the basic steps followed in carrying out the method;

FIG. 13 illustrates apparatus for making an agent-containing film;

FIG. 14 illustrates a number of stents mounted onto a mandrel with a strip of the film formed by the apparatus of FIG. 13 being wound on to the stents to create first stent/film subassemblies;

FIG. 15 illustrates a support and rotating structure used in the winding of the strip of film of FIG. 14;

FIG. 16 is an enlarged view of a section of the first stent/film subassemblies of FIGS. 14 and 15 after a protective layer has been wound over the strip of film of the first stent/film subassemblies to create second stent/film subassemblies;

FIG. 17 illustrates the resulting stent/film combinations mounted on the mandrel after adhering the film to the stent, removing the protective layer and trimming any excess film; and

FIG. 18 illustrates a stent/film combination of FIG. 17 after removal from the mandrel.

### DETAILED DESCRIPTION OF THE INVENTION

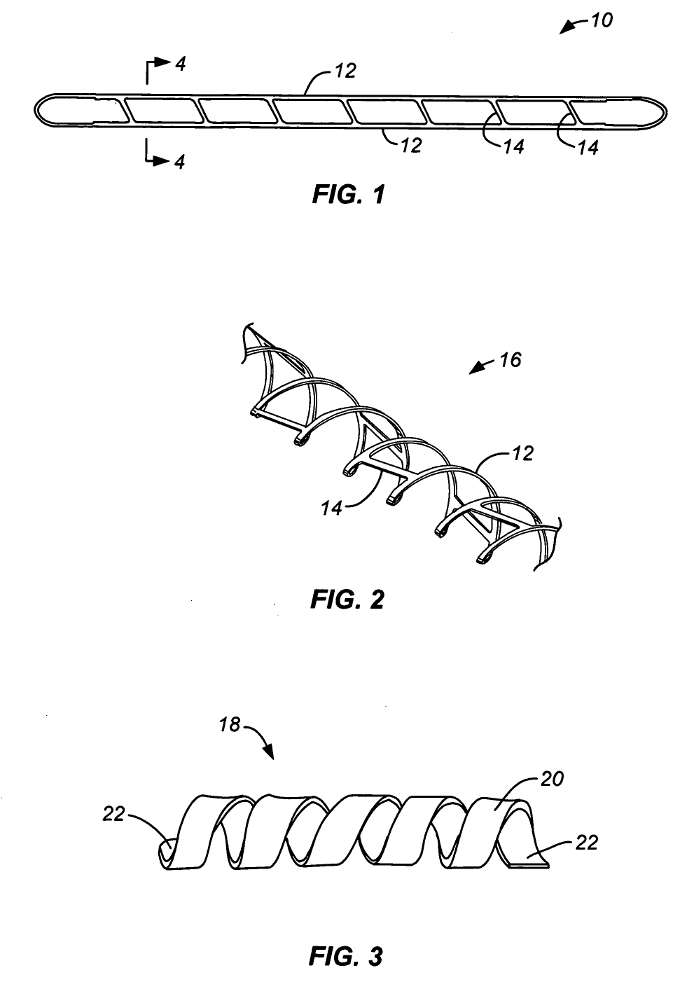

FIG. 1 illustrates a ladder type stent blank 10 having side edges or rails 12 connected by connectors or rungs 14. Stent blank 10 is shown to include two side rails 12; three or more side rail elements may also be used. Stent blank 10 is typically formed into an open spiral as shown in FIG. 2 to create a generally tubular ladder stent 16. Stent blank 10 may also be formed into a tighter wrapped generally tubular spiral so that side rails 12 lie generally adjacent to one another. To create the covered stent 18 of FIG. 3, a sleeve of porous graft-type material 20, such as made of ePTFE, is typically slid over stent blank 10 prior to forming stent blank 10 into the spiral shape of FIG. 2. The ends 22 of material 20 are typically sealed in an appropriate manner, such as by the use of an appropriate adhesive or by using other bonding techniques.

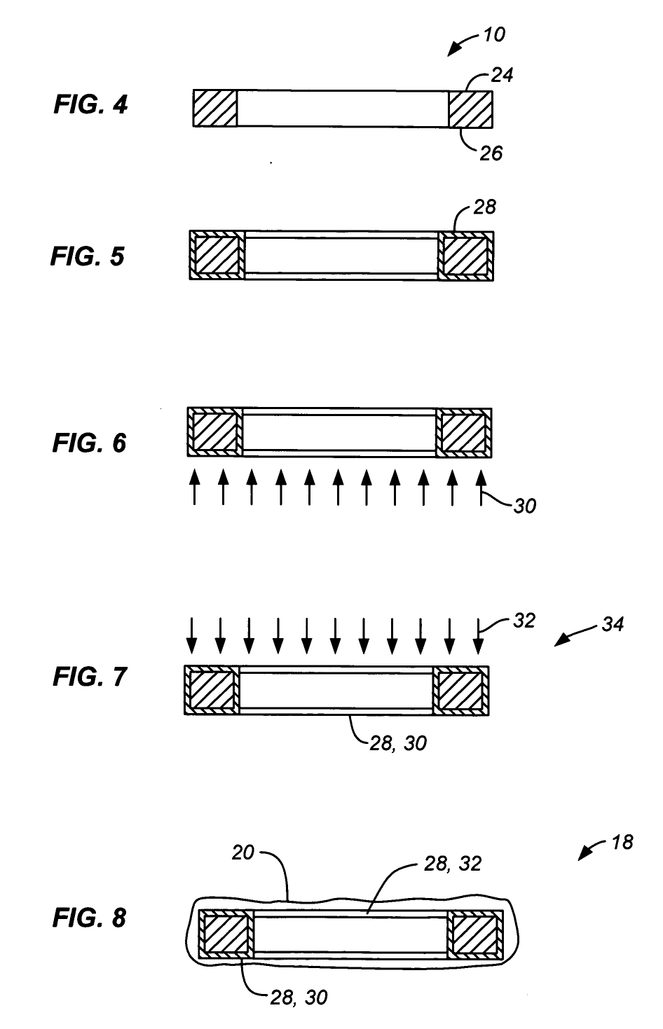

The above-described structure is generally conventional. With the present invention stent blank 10 is treated as discussed with reference to FIGS. 4-7, typically prior to being enclosed within material 20. FIG. 4 is an enlarged cross sectional view of stent blank 10 having an outer stent body surface 24 and an inner stent body surface 26. A therapeutic agent, such as one or more of Sodium Nitroprusside, L-Arginine and Poly L-Arginine, is applied to stent blank 10. This is typically accomplished using a matrix of silicone or other matrix and the therapeutic agent applied as a liquid or semi-solid composition to stent blank 10. The composition is then stabilized, typically cured or polymerized, resulting in stent blank 10 being general uniformly covered with a silicone/therapeutic agent matrix 28. Stent blank 10 need not be uniformly covered but could have the therapeutic agent applied only to outer stent body surface 24. Also, multiple layers of the same, or different, therapeutic agent may be used with stent blank 10. This would provide flexibility in the delivery of one or more therapeutic agents. For example, the agent could be delivered in a multi-modal release with, for example, an initial bolus type delivery followed by at least one extended release phase.

After the application of matrix 28, a diffusion barrier material 30 is applied to at least inner stent body surface 26, and may be applied to all surfaces of stent blank 10 except for outer stent body surface 24. Diffusion barrier material 30 is provided to prevent passage of at least a significant amount of the therapeutic agent within matrix 28 from being diffused therethrough. A preferred diffusion barrier material is Parylene applied as a vapor. The thickness of diffusion barrier material 30 using Parylene is preferably greater than about 3.5 micrometers thick and is typically about 3-5 micrometers thick. At these thicknesses, the Parylene is an effectively uninterrupted later of Parylene and therefore sufficiently nonporous to act as an effective barrier to the passage of the therapeutic agent.

FIG. 7 illustrates application of a diffusion restrictor material 32 to outer stent body surface 24. Material 32 is used to restrict or otherwise control the passage of the therapeutic agent from matrix 28 at surface 24. A preferred diffusion restrictor material is also Parylene applied as a vapor. The thickness of diffusion restrictor material 32 comprising Parylene is preferably less than about 2.5 micrometers thick and is typically about 1-3 micrometers thick. At these thicknesses, material 32 is not an interrupted layer but has pinhole-like openings to create an effectively porous diffusion restrictor. The resulting stent structure 34 comprises stent blank 10 covered by matrix 28 over which diffusion barrier material 30 and diffusion restrictor material 32 have been applied. Thereafter, stent structure 34 is enclosed within material 20, see FIG. 8, and then coiled to create covered stent 18.

Diffusion barrier material 30 and diffusion restrictor material 32 may be made so that barrier material 30 prevents any measurable diffusion of the applicable agent through it while restricting material 32 permits diffusion of the agent at a first, therapeutic rate for the intended therapy. However, barrier material 30 typically allows the diffusion of some of the agent through it, but at a second rate, the second rate being less than the first, therapeutic rate. In one embodiment the second rate is at least 50% less than the first rate. The acceptable percentage will depend on various factors including the therapeutic agent used, the patient's condition, state of the disease, vascular flow, target site, the particular prior therapy, and so forth.

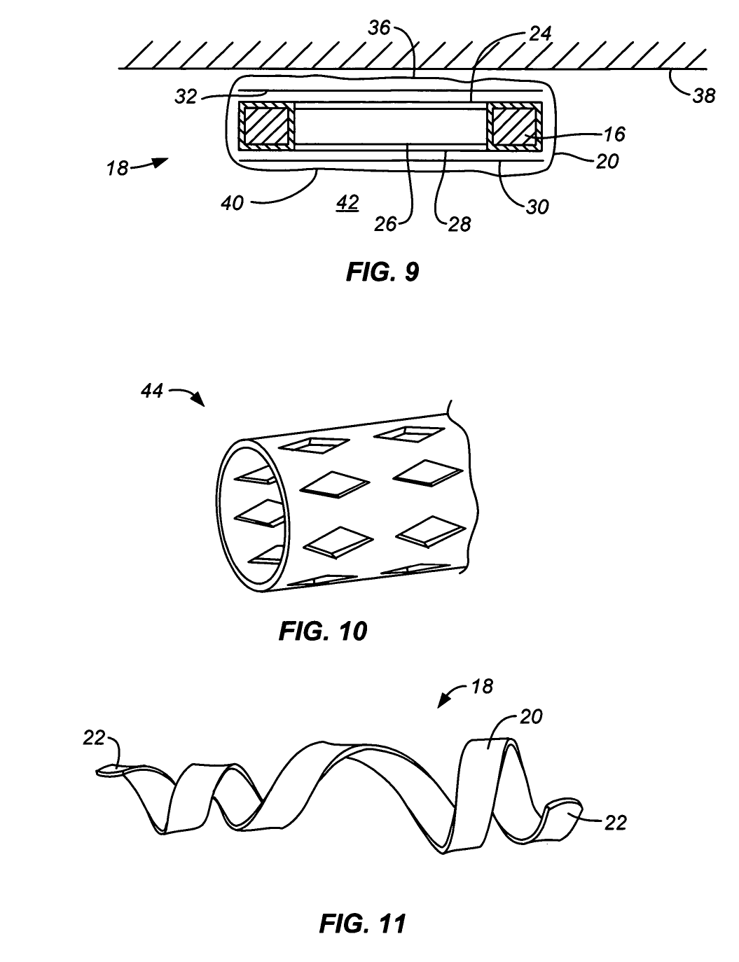

FIG. 9 is a simplified cross sectional view of covered stent 18 similar to that of FIG. 8 with the various layers separated for purposes of illustration. Covered stent 18 is located within a vessel, such as a blood vessel, and with an outer material portion 36 of material 20 being positioned against the vessel wall 38 so that an inner material portion 40 of material 20 faces the open interior 42 of the vessel. Once in place against vessel wall 38, the therapeutic agent within matrix 28 may slowly diffuse through diffusion restrictor material 32 and outer material portion 36 and pass into a vessel wall 38. However, due to the use of diffusion barrier 30, diffusion of the therapeutic agent into interior 42 of the vessel is at least substantially reduced. This helps prevent wasting of the therapeutic agent as well as reducing or eliminating any negative consequences from the introduction of the therapeutic agent into vessel interior 42 and the systemic circulation.

Diffusion barrier material 30 and diffusion restrictor material 32 may be applied elsewhere, for example to the inner surface of inner material portion 40 or the inner surface of outer material portion 36, or both, instead of or in addition to application onto matrix-covered stent blank 10. In such case the therapeutic agent may be relatively loosely contained between diffusion barrier material 30 and stent blank 10 and between diffusion restrictor material 32 and stent blank 10.

The invention has been discussed with reference to a ladder-type stent 16. The invention may also be used with other types of stents, such as a cylindrical, expanded metal stent 44, shown in FIG. 10, having an appropriate sleeve of porous material covering both the inner and outer surfaces (not shown). FIG. 11 illustrates an alternative embodiment of the covered stent 18 of FIG. 3 having a variable pitch, that is different spacing between the turns, and a variable diameter.

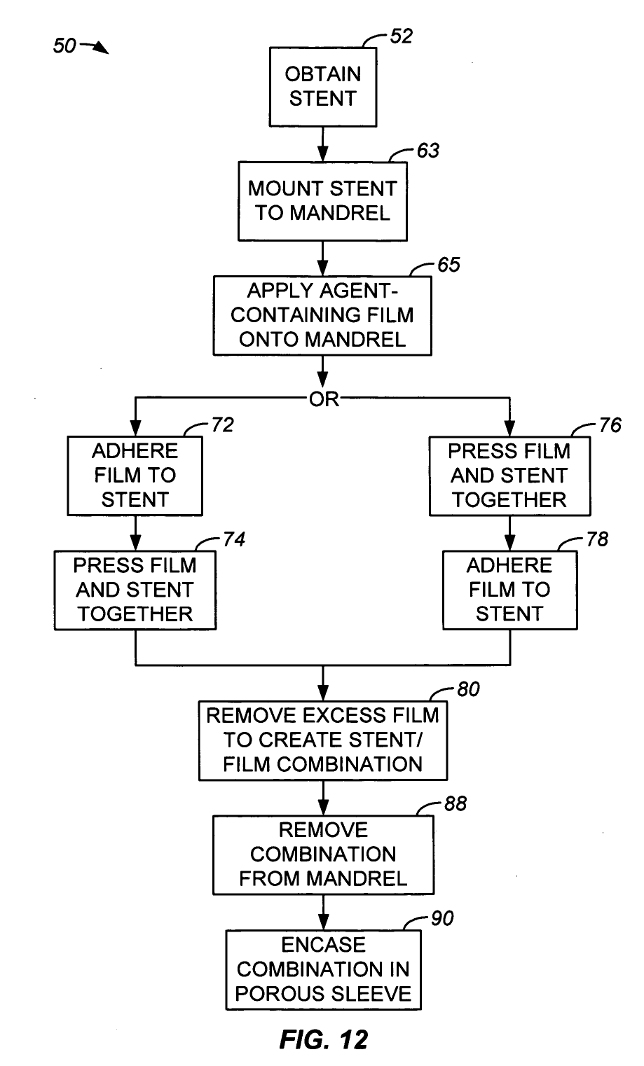

Various methods and techniques for applying an agent-containing matrix material to the stent have been described above. A method 50 for making a covered agent-eluting stent will now be discussed with reference primarily to FIGS. 12-19 with like reference numerals referring to like elements. Method 50 will be useful for stents having openings such as the ladder stent illustrated in FIG. 2. While the ladder stent of FIG. 2 will be referred to in the following discussion, it should be understood that other stents having openings, such as the stent illustrated in FIG. 10, may also be used with this method.

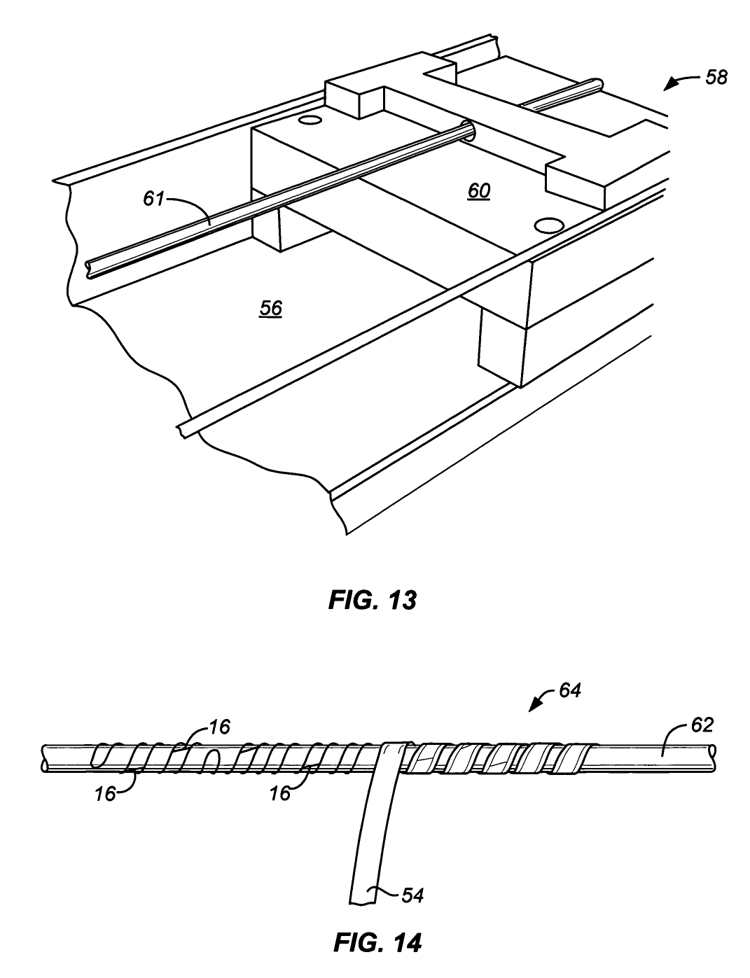

FIG. 12 is a very basic, general flowchart of method 50. It is to be understood that the steps may not necessarily be accomplished in the order indicated in FIG. 12 and that additional steps, as discussed below, will typically be used. A stent 16, such as shown in FIG. 2, having openings defined between rails 12 and rungs 14 is obtained at step 52. An agent containing film 54, see FIG. 14, may then be made by first mixing the agent with, for example, liquid silicone and a volatile vehicle, such as xylene. The mixture is then deposited on the surface 56 of a film making apparatus 58 shown in FIG. 13, typically using a syringe. Surface 56 is typically made of PTFE. Apparatus 58 includes a spreader block 60 that is pulled over surface 56 by the actuation of a drive screw 61. The mixture has an appropriate thickness, typically created by the gap between the spreader block 60 and surface 56. In one embodiment this gap is 0.019 in. (0.5 mm). The spread mixture then at least partially cures to a film sheet which is then sliced into a number of strips of film 54. This curing of the mixture typically takes place as the xylene or other volatile vehicle dissipates. In some situations, as discussed below, film 54 is desired to be fully cured (typically about two hours) before it is used while in other situations it is desired that film 54 be partially cured when it is used to increase the adhesive characteristics of the film.

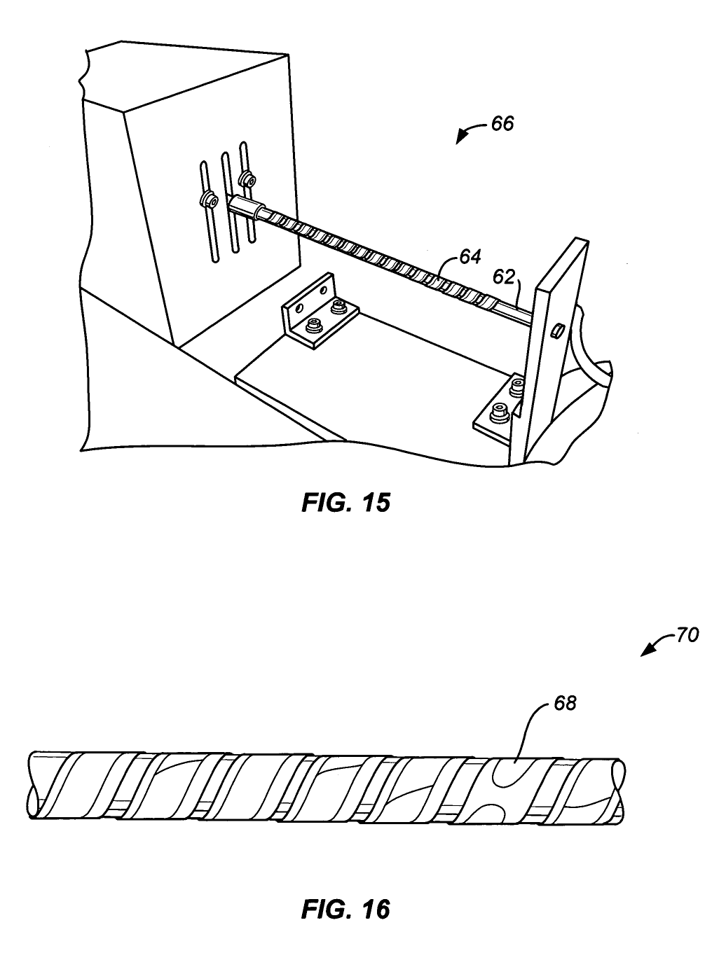

FIG. 14 illustrates a number of stents 16 mounted onto a mandrel 62, see step 63 in FIG. 12, after which a strip of film 54 is wound on to the stents, see step 65 in FIG. 12, to create first stent/film subassemblies 64. It is preferred that the outside diameter of mandrel 62 is somewhat larger than the inside diameter of stents 16 when in the relaxed state to help ensure the stents remain in good contact with the mandrel. As shown in FIG. 15, a support and rotating structure 66 is used in the winding of the strip of film of FIG. 14.

FIG. 16 is an enlarged view of a section of the first stent/film subassemblies 64 after a protective layer 68 has been wound on top of the strip of film 54 of the first stent/film subassemblies in preparation for making second stent/film subassemblies 70. Protective layer 68 is typically a material such as FEP (fluorinated ethylene propylene). Protective layer 68 preferably has elastic properties that allow it to be wound onto film 54 to press film 54 against stent 16. At this point the processing may follow steps 72 and 74 or steps 76 and 78 as indicated in FIG. 12. Although in this disclosed embodiment protective layer 68 is a relatively long, thin strip of material similar to film 54, either or both of layer 68 and film 54 could, in appropriate circumstances, be much wider having, for example, a width extending the entire length of each stent 16 on mandrel 62.

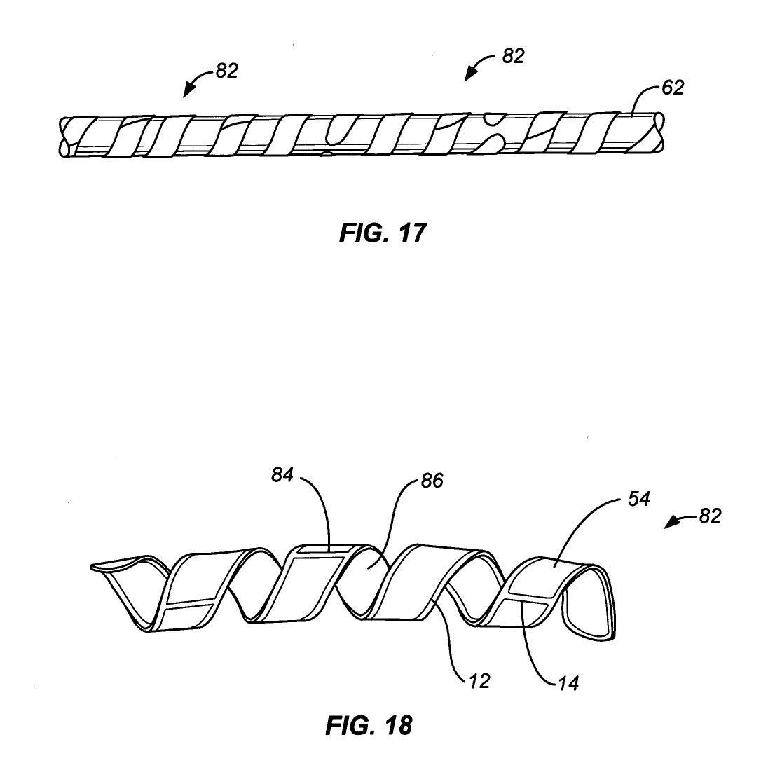

Steps 72 and 74 are followed when the surface of film 54 is sufficiently adhesive to adhere to stent 16. One way of achieving this is to partially cure film 54 so that one side of the film, typically the side of the film contacting surface 56, has sufficient adhesive properties relative to stent 16 so that no additional adhesive is required. Alternatively, stent 16 could be coated with adhesive or at least one side of film 54 could be coated with an adhesive, or both. In addition, film 54 could be partially cured and an additional adhesive could also be used. At step 74, film 54 and stent 16 are pressed together, such as by rolling the structure of FIG. 16 over a flat surface using moderate hand pressure. This causes film 54 to be pressed into the openings in stent 16 with film 54 adhering to the stent but not to protective layer 68. Thereafter, which may include a delay to reduce any adherence of protective layer 68 and film 54, protective layer 68 is removed from pressed film 54 and stent 16. This exposes pressed film 54 to permit any excess film to be trimmed or otherwise removed pursuant to step 80 of FIG. 12 to create stent/film combinations 82 as shown in FIG. 17. The resulting stent/film combinations 82 are then removed from mandrel 62, step 88 of FIG. 12. One such stent/film combination 82 is shown in FIG. 18. As can be seen in FIG. 18, film 54 is adhered to stent 16 and fills the openings defined by rungs 14 and rails 12.

If it is desired to provide a diffusion restrictor to the outer surface 84 of combination 82 and a diffusion barrier to the inner surface 86 of combination 82, combination 82 may be enclosed with a shrink wrap film to cover outer surface 84 and then apply, for example, Parylene through vapor deposition within a vacuum chamber, typically at room temperature, thereby applying a layer of Parylene to inner surface 86. Then the shrink wrap film is removed and combination 82 is again placed in a vapor deposition vacuum chamber to apply Parylene, or some other material, to both outer and inner surfaces 84, 86. Assuming, for example, the deposition rate and time are the same for both deposition steps, then there will be twice as much Parylene deposited on inner surface 86 as on outer surface 84. Therefore, the Parylene layer on outer surface 84 can act as a diffusion restrictor while the Parylene layer on inner surface 86 can act as a diffusion barrier. This 2 to 1 thickness ratio can be changed. Also, different materials can be used to create diffusion restrictors and diffusion barriers. Different methods can be used to apply the diffusion restrictors and diffusion barriers.

Instead of proceeding along steps 72 and 74, the process may proceed along steps 76 and 78. According to this aspect of the invention, film 54 is typically cured so that it does not have a surface sufficiently adhesive to adhere to stent 16. However, it has been found that it is better to place the side of film 54 that contacted surface 56 against stent 16 because it is tackier than the opposite side. During the film/stent pressing step 76, second subassemblies 70 on mandrel 62 are typically placed on a hard surface and a relatively heavy metal block is rolled over this combination to cause rails 12 and a rungs 14 of stent 16 to cut into film 54 to cause the film to enter the open areas bounded by the rails and rungs and create second subassemblies 70. Thereafter protective layer 68 is removed and an adhesive, typically the same mixture of the agent, liquid silicone and a volatile vehicle, such as xylene, as used to create film 54, is painted or otherwise applied onto film 54. A second protective layer 68 is then placed over adhesive-covered film 54 and allowed to cure, typically 2-4 hours or overnight. The second protective layer 68 is then removed and the structure is allowed to dry, typically four hours or overnight. The process continues as described above starting with step 80 to create combination 82 of FIG. 18.

Finally, combination 82, created according to either procedure, is enclosed within a sleeve of porous material 20 to create a covered, agent-eluting stent 18, such as shown in FIGS. 3 or 11. See step 90 of FIG. 12. When the agent is Sodium Nitroprusside, the ratio by weight of Sodium Nitroprusside to silicone for combination 82 is typically about 40% Sodium Nitroprusside to 60% silicone. However, the expected practical limits for the percentage of Sodium Nitroprusside ranges from a maximum of about 60% to a minimum of about 5%.

In some situations it may be desired to provide an initial bolus of the agent. One way to do so is to apply another layer of the same mixture as used to create film 54 over the Parylene-covered outer surface 84 of combination 82. The bolus layer will, compared to film 54, typically be a thinner layer with a lower percentage of agent to silicone.

Another alternative is the use of two films 54. A first film 54 would be wrapped on mandrel 62, stent 16 would be mounted on to the mandrel and over the first film, and a second film 54 would be wrapped on top of the stent. If the opposed sides of the first and second films 54 are sufficiently tacky to provide good adhesion to one another and to stent 16, a separately applied adhesive will not be needed. Otherwise, a separate adhesive may be applied to one or more of stent 16 and the two films 54. After covering with a protective layer 68, the processing steps may proceed as discussed above. It is believed that this procedure, as well as the procedure discussed above with regard to steps 72 and 74, provide better distribution of the agent as compared with the procedure described with regard to steps 76 and 78.

Other modification and variation can be made to the disclosed embodiments without departing from the subject of the invention as defined in following claims. For example, adhering to the film to the stent may take place by subjecting the film and stent to, for example, electromagnetic energy, ultrasound energy, heat, or other external influences to cause adhesion between the two.

Any and all patents, patent applications and printed publications referred to above are incorporated by reference.

What is claimed is:

1. A method for making a covered agent-eluting stent comprising:
obtaining a stent having a stent body with openings formed therein;
mounting the stent onto a mandrel;
applying an agent-containing film onto the stent mounted on the mandrel to create a first subassembly;
pressing the stent and the film against one another to create a second subassembly with at least a portion of the film pressed at least partially into the openings of the stent body;
adhering the film to the stent;
removing any excess film from the second subassembly to create a stent/film combination having inner and outer surfaces;
removing the stent stent/film combination from the mandrel; and
enclosing the stent/film combination within a sleeve of porous material to create a covered agent-eluting stent.

2. The method according to claim 1 wherein the obtaining step comprises obtaining a coiled stent.

3. The method according to claim 1 wherein the obtaining step comprises obtaining a coiled stent having axially spaced-apart turns to define a generally helical gap between the turns.

4. The method according to claim 2 wherein the obtaining step comprises obtaining a coiled stent having an inside diameter when in a relaxed state.

5. The method according to claim 4 further comprising selecting a mandrel having an outside diameter larger than the inside diameter of the coiled stent.

6. The method according to claim 1 wherein the applying step comprises winding a strip of the agent-containing film onto the stent.

7. The method according to claim 1 further comprising:
preparing a flowable agent-containing mixture, the mixture comprising an agent and a matrix material;
spreading the mixture onto a surface; and
at least partially curing the spread mixture to create the agent-containing film.

8. The method according to claim 7 wherein the preparing step is carried out using silicone as the matrix material with the mixture comprising a volatile vehicle;

9. The method according to claim 1 wherein:
the adhering step is carried out before the pressing step; and
the adhering step is carried out using a film having an adhereable surface contacting the stent during the applying step.

10. The method according to claim 9 wherein the adhering step is carried out using a partially cured film.

11. The method according to claim 9 wherein the adhering step is carried out using an adhesive applied to at least one of the film and the stent.

12. The method according to claim 9 further comprising applying a protective layer to the first subassembly before the pressing step and removing the protective layer from the second subassembly after the pressing step.

13. The method according to claim 1 wherein:
the adhering step is carried out after the pressing step; and
the adhering step comprises applying an adhesive to the second subassembly.

14. The method according to claim 13 further comprising:
applying a first protective layer to the first subassembly before the pressing step and removing the first protective layer from the second subassembly after the pressing step; and
applying a second protective layer to the second subassembly after the pressing step and removing the second protective layer from the second subassembly after the second protective layer applying step.

15. The method according to claim 1 wherein they stent removing step is carried out after the excess film removing step.

16. The method according to claim 1 further comprising:
applying a diffusion restrictor on the outer surface of the stent/film combination, said diffusion restrictor permitting passage of the agent through the diffusion restrictor at a first, therapeutic rate; and
applying a diffusion barrier on the inner surface of the stent/film combination, said diffusion barrier preventing passage of the agent through the diffusion barrier at at most a second rate, the second rate being less than the first rate.

17. The method according to claim 16 wherein the applying steps are carried out after the removing step.

18. The method according to claim 16 wherein the diffusion barrier applying step is carried out to create a substantially non-porous vapor-deposited layer of Parylene and the diffusion restrictor applying step is carried out to create a micro-porous vapor-deposited layer of Parylene.

19. The method according to claim 16 further comprising applying a bolus-creating agent-containing material on the diffusion restrictor.

20. The method according to claim 1 wherein the applying step is carried out with an agent-containing film comprising a hydrophilic agent.

21. A method for making a covered agent-eluting stent comprising:
obtaining a coiled stent having a stent body with openings formed therein, the stent having axially spaced-apart turns to define a generally helical gap between the turns;
mounting the stent onto a mandrel;
winding a strip of an agent-containing film onto the stent mounted on the mandrel to create a first subassembly;
pressing the stent and the film against one another to create a second subassembly with at least a portion of the film pressed at least partially into the openings of the stent body;
adhering the film to the stent;
removing any excess film from the stent to create a stent/film combination having inner and outer surfaces;
removing the stent/film combination from the mandrel;
applying a diffusion restrictor on the outer surface of the stent/film combination, said diffusion restrictor permitting passage of the agent through the diffusion restrictor at a first, therapeutic rate;
applying a diffusion barrier on the inner surface of the stent/film combination, said diffusion barrier preventing passage of the agent through the diffusion barrier at at most a second rate, the second rate being less than the first rate;
applying a bolus-creating agent-containing material on the diffusion restrictor; and
enclosing the stent/film combination with the diffusion restrictor, the bolus-creating agent-containing material and the diffusion barrier applied thereto within a sleeve of porous material to create a covered agent-eluting stent.
